(19) United States
(12) Patent Application Publication
(10) Pub. No.: US 2003/0224046 A1
(43) Pub. Date: Dec. 4, 2003

(54) Unit-dose combination composition for the simultaneous delivery of a short-acting and a long-acting oral hypoglycemic agent

(76) Inventors: Vinay Rao, Aurangabad (IN); Dilip G. Saoji, Aurangabad (IN); Harshal P. Bhagwatwar, Aurangabad (IN); Milind C. Shukla, Aurangabad (IN); Noel J. DeSouza, Mumbai (IN)

Correspondence Address: KENYON & KENYON, ONE BROADWAY, NEW YORK, NY, 10004, US

(21) Appl. No.: 10/334,315

(22) Filed: Dec. 31, 2002

Related U.S. Application Data
(60) Provisional application No. 60/385,786, filed on Jun. 3, 2002.

(51) Int. Cl. A61K 9/22; A61K 31/155

(52) U.S. Cl. 424/468; 514/635

(57) ABSTRACT

A stable unit-dose combination composition for the simultaneous delivery of a short-acting oral hypoglycemic biologically active agent (such as, for example, repaglinide or nateglinide), and a long-acting oral hypoglycemic biologically active agent (such as, for example, metformin). Such a composition can be used for the treatment of non-insulin dependent diabetes mellitus (NIDDM) and the improvement of glycemic control.

### REFERENCE TO RELATED APPLICATION

This application claims the benefit of U.S. Provisional Application No. 60/385,786, filed on Jun. 3, 2002.

### FIELD OF THE INVENTION

The present invention relates to a stable unit-dose combination composition for the simultaneous delivery of a short-acting oral hypoglycemic biologically active agent, and a long-acting oral hypoglycemic biologically active agent. Such a composition can be used for the treatment of non-insulin dependent diabetes mellitus (NIDDM) and the improvement of glycemic control. The present invention also relates to processes for the preparation of such combination unit-dose compositions and the use of such combination compositions in the treatment of NIDDM.

### BACKGROUND OF THE INVENTION

Diabetes mellitus is a progressive metabolic disorder in human beings characterized by hyperglycemia and insulin resistance, and is often associated with other disorders such as obesity, hypertension, hyperlipidemia, as well as complications such as cardiovascular disease, retinopathy, and nephropathy. These underlying defects lead to a classification of diabetes into two major classes: (1) Insulin dependent diabetes mellitus (IDDM or Type I diabetes)—where the patients lack β-cells in the pancreas, and such patients are treated with insulin; and (2) Non-insulin dependent diabetes mellitus (NIDDM or Type II diabetes)—where the patients possess β-cells with impaired insulin secretion function.

NIDDM can often be controlled initially by diet and exercise in the initial stages of the disease, but generally requires treatment with injections of exogenous insulin either alone or in combination with one of the following classes of antidiabetic agents to help in the control of blood glucose as the degeneration progresses:

(a) Biguanides, represented principally by metformin, phenformin and buformin, act by decreasing hepatic glucose production as well as intestinal absorption of glucose;

(b) Sulfonylureas, represented principally by glipizide, glimiperide, glyburide, glibornuride, glisoxepide, gliclazide acetohexamide, chlorpropamide, tolazamide, and tolbutamide and others, act by stimulating the release of insulin from the pancreas;

(c) Glitazones, represented principally by rosiglitazone, troglitazone and pioglitazone among others, act by increasing the sensitivity of insulin receptors in the body and diminish or eliminate the need for exogenous insulin; and

(d) Alpha-glucosidase inhibitors, represented principally by acarbose and miglitol, among others, which act by delaying absorption of dietary carbohydrates.

All of the above mentioned antidiabetic agents fall under the category of long-acting orally active hypoglycemic agents (LOAHA). The term “LOAHA” as used herein is intended to designate a hypoglycemic agent with the use of which a maximum secretion of insulin is attained more than 1 hour after administration of the agent. Though all of these treatment modalities have had fair success in controlling NIDDM, none of these therapeutic modalities allow patients the flexibility of choosing the timings of their major meals, viz., breakfast, lunch and dinner.

Recently, repaglinide (PRANDIN™ tablets or NOVONORM™ tablets, Novo Nordisk) and nateglinide (Ajinomoto) which are short-acting orally active hypoglycemic agents (SOAHA) from the meglitinide class of compounds, were introduced into the market for the treatment of NIDDM. These compounds are also insulin secretagogues similar to the sulfonylureas but act at a different site than the sulfonylureas. The term “SOAHA” as used herein is intended to designate a hypoglycemic agent with the use of which a maximum secretion of insulin is attained in less than 1 hour after administration of the agent and the agent is excreted from the body rapidly. These compounds thus find use in the treatment of meal-associated hyperglycemia, i.e. the elevation of blood glucose with the ingestion of food. The rapid acting nature of these compounds allows a patient to tailor his/her antidiabetic treatment around the ingestion of meals. Thus, a patient can take a tablet immediately before, during or after a major meal, preferably within 30 minutes of the meal.

The monograph for PRANDIN™ tablets in the Physicians Desk Reference (PDR 2002, page 2433) describes the use of repaglinide alone or in combination with a sulfonylurea or biguanide where a patient is poorly controlled on either of the compounds alone. The use of repaglinide along with metformin has been demonstrated to be synergistic in controlling NIDDM related symptoms when compared with the use of either of the compounds alone. International patent application WO 01/32158 A2 describes the use of combinations of metformin with other antidiabetic agents such as sulfonylureas, glitazones or repaglinide in treating NIDDM in patients who are drug naive at doses of either or both compounds which are much lower than generally used in patients stabilized on these medicaments individually. The term “drug naive” as used in the application is intended to mean patients who have not been treated with oral antidiabetics. The application demonstrates the preparation of combination unit-dose compositions containing metformin and glyburide and the use of such combinations in providing synergistic efficacy over the compounds used individually as a first-line treatment for drug naive patients. However, the application does not describe the preparation of combination unit-dose combinations containing repaglinide and metformin nor does it demonstrate the use of such combinations in providing synergistic efficacy over the compounds used individually.

International patent application WO 98/56378 and European patent application EP 1097710 A2 describe a novel regimen for the treatment of NIDDM in patients poorly controlled on metformin alone comprising administration of metformin and repaglinide together. These patent applications demonstrated a synergistic effect of this novel combination (administered as the individual marketed products together) over each of the compounds administered alone. However, although the applications claim a unit-dose combination comprising metformin and repaglinide in the same composition, either as a tablet or a capsule, no examples have been provided therein for the preparation of such a composition.

None of the patent applications described above describe the preparation of a unit-dose composition comprising a LOAHA, such as metformin, along with a SOAHA, such as repaglinide, which is physically and chemically stable and releases each drug from the unit-dose composition at a rate similar to that compared to the individually marketed products (such as, for example, repaglinide from PRANDIN™ tablets or NOVONORM™ tablets (Novo Nordisk), and metformin from GLYCIPHAGE™ tablets (Franco Indian)). Furthermore, none of the applications describe a unit-dose combination composition comprising a controlled release LOAHA, such as a biguanide (metformin), and an immediate release SOAHA, such as repaglinide, for the treatment of NIDDM in patients already stabilized but poorly controlled by controlled-release metformin alone. Also, similar combinations of metformin (biguanide) with nateglinide have not been described.

A chemically and physically stable dosage form which can provide therapeutic levels of a metformin and repaglinide or nateglinide from the same unit-dose composition in a fashion similar to each of the separate products available commercially would be extremely beneficial in clinical practice for glycemic control in the treatment of NIDDM for patients poorly controlled on either metformin or repaglinide/nateglinide alone. Such a dosage form could then be administered twice or thrice a day as required by the patient resulting in enhanced patient compliance. Furthermore, such a physically and chemically stable dosage form comprising a controlled-release biguanide, such as metformin, and an immediate release repaglinide or nateglinide would allow a physician the flexibility of administering both of these active agents together to patients already stabilized but poorly controlled by controlled-release metformin. This would provide enhanced basal insulin levels because of the prolonged and controlled action of controlled-release metformin which would be further enhanced by the short action of repaglinide or nateglinide in providing improved glycemic control.

Thus, there is a need for an immediate release unit-dose combination composition comprising metformin and repaglinide for the treatment of NIDDM in patients poorly controlled on either metformin or repaglinide alone.

There is also a need for an immediate release unit-dose combination composition comprising metformin and nateglinide for the treatment of NIDDM in patients poorly controlled on either metformin or nateglinide alone.

There is a further need for a unit-dose combination composition comprising a controlled release metformin and an immediate release repaglinide for the treatment of NIDDM in patients poorly controlled on either metformin or repaglinide alone.

There is still a further need for a unit-dose combination composition comprising a controlled release metformin and an immediate release nateglinide for the treatment of NIDDM in patients poorly controlled on either metformin or nateglinide alone.

There is also a need for such compositions which are physically and chemically stable.

There is a further need for such a composition which releases both the metformin and repaglinide or nateglinide at a rate which is substantially similar to that of each of the individually marketed products in individual form.

There is also a need for a method of use of such a combination for the treatment of NIDDM in patients poorly controlled by either metformin or repaglinide/nateglinide alone.

### SUMMARY OF THE INVENTION

The present invention is directed to an immediate release unit-dose composition comprising metformin along with repaglinide or nateglinide, which is physically and chemically stable, and releases each drug from the unit-dose composition at a rate similar to that of the individually marketed products in individual form (viz., repaglinide from formulations such as PRANDIN™ tablets or NOVONORM™ tablets (Novo Nordisk), and metformin from formulations such as GLYCIPHAGE™ tablets (Franco Indian)).

The present invention is also directed to unit-dose compositions comprising a controlled-release metformin along with an immediate release repaglinide or nateglinide.

Furthermore, the present invention is also directed to processes for the preparation of such compositions and the use of such compositions in the treatment of NIDDM.

### BRIEF DESCRIPTION OF THE DRAWINGS

FIG. 1 shows the dissolution profile of repaglinide from NOVONORM™ (Novo Nordisk) 2.0 mg tablets as compared to from repaglinide 2.0 mg+metformin HCl 500 mg tablets (Wockhardt).

FIG. 2 shows the dissolution profile of metformin HCl from GLYCIPHAGE™ (Franco Indian) 500 mg tablets as compared to from repaglinide 2.0 mg+metformin HCl 500 mg tablets (Wockhardt).

### DETAILED DESCRIPTION OF THE INVENTION

This invention is directed to antidiabetic unit-dose combination compositions, and processes for the preparation of such compositions, for the simultaneous immediate release of a low-dose (about 0.2 to about 200 mg for repaglinide or nateglinide), low-aqueous solubility (insoluble, or 1 part of solute soluble in 10,000 parts of solvent or greater), short-acting, orally active, hypoglycemic, antidiabetic agent (such as, for example, repaglinide), and the immediate or controlled release of a high-dose (about 200 to about 1200 mg), high-aqueous solubility, long-acting, orally active, hypoglycemic, antidiabetic agent (such as, for example, a biguanide such as metformin hydrochloride). This is a particularly challenging task for the pharmaceutical formulation scientist because of issues such as the uniformity of content of the low-dose agent in the matrix, and the physical and chemical stability of both the active agents in such a unit-dose composition.

The biguanides that could be used in the processes and compositions of this invention include metformin, phenformin, buformin and other medicinally active and pharmaceutically acceptable forms from the biguanide class of compounds, their salts, solvates, hydrates, polymorphs, complexes and other such products. A particularly preferred biguanide is metformin because of its proven clinical use. Different salts of metformin that could be used in the present invention include hydrochloride, acetate, maleate, fumarate, succinate and other salts. A detailed description of the different salts of metformin is described in the literature and is available in U.S. Pat. No. 6,031,004, which is herein incorporated by reference in its entirety. As would be understood by one of ordinary skill in the art, the same or similar salts could be prepared for buformin, phenformin and other compounds from the biguanide class of compounds. These same or similar salts could also be used in accordance with the present invention.

The metformin hydrochloride or pharmaceutically acceptable salts of metformin are preferably present in an amount of from about 30% to about 95% by weight, more preferably from about 55% to about 70% by weight of the total composition.

The meglinitides that could be used in accordance with the present invention include repaglinide, nateglinide, and other medicinally active and pharmaceutically acceptable forms from the sulfonylurea class of compounds, their salts, solvates, hydrates, polymorphs, complexes and other such products. The preferred compounds are repaglinide and nateglinide.

The repaglinide, nateglinide or their pharmaceutically acceptable salts are preferably present in an amount of from about 0.05% to about 5.0% by weight, more preferably from about 0.1% to about 1.0% by weight of the total composition.

The combination immediate-release unit-dosage formulation can be prepared in the forms which are well-known in the art, such as, for example, a tablet or a capsule form. When a tablet dosage form is prepared the metformin and the repaglinide or nateglinide can be intimately mixed with one another in the matrix of the tablet or a bilayered tablet can be prepared. The term “bilayered” as used herein is intended to encompass formulations where two separate layers are prepared by the compression of individual granules containing the two active agents, or one active agent is present in a coating which is formed on a core containing the second active agent.

A capsule dosage form for such a combination can contain a simple blend of the two active agents with the addition of suitable excipients or non-pareil sugar seeds and the like coated with the active ingredients filled into the capsule shells. Of course, the use of a tablet of one active and a powder or granules of the other active both filled into a capsule is well within the scope of the present invention.

The excipients to be used in the preparation of an immediate release unit-dose combination can be chosen from those routinely used in the art of preparation of pharmaceutical solid dosage forms. Such excipients include, but are not limited to, binding agents, bulking agents, disintegrants, glidants, wetting agents, lubricating agents, pigments, dyes and the like and are known to persons skilled in the art of developing and manufacturing pharmaceutical solid oral dosage forms. The choice of the capsule shells and the way the active materials are filled into the capsule shells, as well as the choice of the tablet shape and size can be chosen by a person skilled in the art of preparation of pharmaceutical solid oral dosage forms.

One embodiment of the present invention includes a combination composition wherein the two active agents are intimately dispersed in the same tablet matrix. It is a particular challenge to a pharmaceutical scientist to develop such a formulation where the low-dose low-solubility repaglinide is uniformly dispersed in the high-dose highly water-soluble metformin and is released at the desired rate.

Thus, one of the essential components of such a combination unit-dose composition is a wetting agent. The wetting agent is required for the proper wetting of the highly water-insoluble repaglinide and to allow its rapid and complete release from the final matrix. The wetting agent also aids in the uniform distribution of the repaglinide within the metformin matrix. The wetting agent could be chosen from surfactants, emulsifiers, bile salts, phospholipids and such other materials known to possess properties for wetting enhancement. Handbook of Pharmaceutical Excipients (1994), Handbook of Pharmaceutical Additives (1995) and international patent application WO99/42016 provide a more detailed listing of different emulsifiers useful in pharmaceutical formulations and are all incorporated herein by reference in their entireties. Of course, combinations of the above described wetting agents could also be used to enhance the wetting of the active agents or to reduce the actual particle size of the active medicament through surface solubilization. A more detailed description of the different wetting agents which can be used in accordance with the compositions of the present invention is provided in U.S. Pat. No. 6,248,363 to Patel et al., which is incorporated herein by reference in its entirety.

The combination unit-dose tablet of the present invention can be prepared and coated by processes known to persons skilled in the art of manufacturing solid oral dosage forms. The optional coating, when present, is generally water-soluble and should dissolve rapidly when administered to the patient, preferably within 5 minutes of ingestion.

In a representative embodiment of the composition of the present invention, a combination unit-dose combination comprising an immediate release repaglinide or nateglinide and a controlled-release metformin can be prepared simply by coating a controlled-release metformin core tablet with a coating containing the repaglinide or nateglinide dispersed therein, with or without a wetting agent as described above. The controlled-release metformin core tablet could be any of those described in the literature, such as, for example, an osmotic tablet as described in U.S. Pat. No. 6,099,859, a matrix tablet as described in U.S. Pat. No. 5,955,106, or a biphasic release tablet as described in international patent application WO 99/47128. Of course, any formulation which can provide a controlled release of the metformin over the desired time period based on a variety of mechanisms, such as, for example, diffusion, disintegration, osmotic behavior, swelling, erosion, or combinations of any of these, are within the scope of the present invention.

The combination unit-dose compositions of the present invention preferably contain from about 0.25 mg to about 5 mg of repaglinide and from about 200 mg to about 1200 mg of metformin. More preferably, the combination unit-dose compositions contain from about 0.5 mg to about 2.0 mg of repaglinide and from about 500 mg to about 1000 mg of metformin.

When a combination unit-dose composition containing nateglinide is to be prepared in accordance with the present invention, it preferably contains from about 20 to about 200 mg of nateglinide and from about 200 to about 1200 mg of metformin. More preferably, the combination unit-dose composition contains from about 60 mg to about 180 mg of nateglinide and from about 400 mg to about 1000 mg of metformin. Of course, where a controlled-release metformin is involved, the dose range of metformin could be reduced further to from about 300 mg to about 600 mg, more preferably to about 500 mg for controlled-release metformin.

It is to be understood, however, that for any particular subject being treated (e.g., a mammal), specific dosage regimens should be adjusted according to the individual need. Thus, a unit-dose composition comprising about 0.5, 1 or 2 mg of immediate-release repaglinide along with about 500, 850 or 1000 mg of immediate- or controlled-release metformin in the formulation is within the scope of the present invention. Similarly, a unit-dose composition comprising about 60, 120 or 180 mg of immediate-release nateglinide along with about 500, 850 or 1000 mg of immediate- or controlled-release metformin in the formulation is within the scope of the present invention. It is further to be understood that the dosages set forth herein are exemplary only and they do not to any extent limit the scope of the present invention.

In addition, the compositions of the present invention show a dissolution profile substantially similar to that of the individually marketed products (as shown in the examples described herein).

### EXAMPLES

Specific preferred embodiments of the invention will now be described with reference to the following examples which should be regarded in an illustrative rather than a restrictive sense.

### Example 1

TABLE 1
Unit-Dose Composition Formula for Repaglinide 2.0 mg & Metformin
Hydrochloride IP 500 mg Tablets

| | | Quantity per tablet |
|---|---|---|
| No. | Ingredient | (mg) |
| 1. | Metformin hydrochloride (active ingredient) | 500.00 |
| 2. | Repaglinide (active ingredient) | 2.00 |
| 3. | Lutrol F 68 (wetting agent) | 5.00 |
| 4. | Microcrystalline cellulose (bulking agent) | 100.00 |
| 5. | Polyvinyl pyrollidone (binding agent) | 32.00 |
| 6. | Colloidal silicon dioxide (wicking agent) | 3.00 |
| 7. | Magnesium Stearate (lubricant) | 7.00 |
| 8. | Talc (glidant) | 3.00 |
| 9. | Hydroxypropyl methylcellulose (coating | 2.00 |
| | agent) | |
| 10. | Titanium dioxide (opacifier) | 0.50 |
| 11. | PEG-400 (plasticizer) | 0.30 |
| 12. | FD & C Yellow No. 2 (colorant) | 0.50 |

The tablet with the composition formula listed above in Table 1 is prepared as described below in the method of Example 2.

### Example 2

### Method of Manufacture for Repaglinide & Metformin Hydrochloride Tablets

Metformin hydrochloride, and Lutrol F68 were passed through a 30 mesh sieve and transferred to a suitable blender followed by mixing for 10 minutes to obtain a dry blend. Repaglinide was dissolved in a mixture of methylene chloride and isopropyl alcohol (1:10, v/v) containing polyvinyl pyrrolidone of viscosity grade K30 to form a drug-binder solution. The dry blend was further granulated with the drug-binder solution. The granules were air-dried to below 1.5% loss on drying. The blend was lubricated with microcrystalline cellulose, magnesium stearate, talc and colloidal silicon dioxide. The granules were compressed into tablets on a compression machine followed by film coating.

### Example 3

### Content Uniformity of the Novel Unit-Dose Combination Composition

The compressed tablets prepared in Example 1 were tested as per the method described in the United States Pharmacopoeia, USP 25, pages 2083-84, which is herein incorporated by reference, for the content uniformity of repaglinide. The tablets demonstrated an assay of repaglinide of 101.32% of label claim (2 mg per tablet) with a uniformity of content of 100%±1.33% and an assay of metformin of 99.57% of label claim (500 mg per tablet). This example demonstrates that tablets with excellent uniformity of content of the low-dose low-solubility SOAHA (repaglinide in this example) in the high-dose high-solubility LOAHA (metformin in this example) could be prepared.

### Example 4

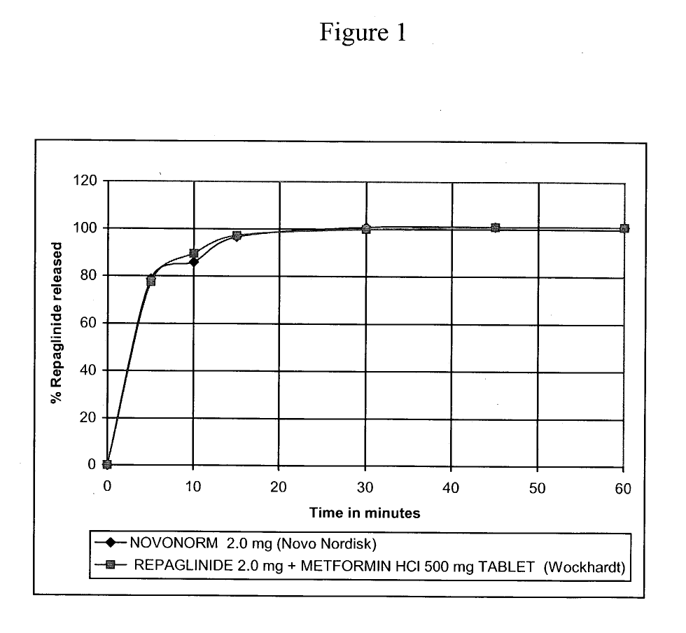

In this example, we compared the dissolution profile of the unit-dose combination composition of the present invention to the dissolution profiles of each of the individually marketed products contained therein. That is, as can be seen in table 2 below, we first compared the dissolution profile for repaglinide from the unit-dose combination composition of the present invention (repaglinide+metformin (Wockhardt)) to that of repaglinide from its individually marketed form (NOVONORM™ (Novo Nordisk)). As shown in FIG. 1, which plots the data from table 2, the unit-dose combination composition of the present invention releases the repaglinide at a substantially similar rate to that of the individually marketed product.

TABLE 2
Comparative Dissolution Profile for Repaglinide From
NOVONORM ™ 2.0 mg (Novo Nordisk) and Repaglinide
2.0 mg + Metformin HCl 500 mg Tablets (Wockhardt)

| | Time | NOVONORM ™ | Repaglinide + Metformin, |
|---|---|---|---|
| No. | (min) | (Novo Nordisk) | (Wockhardt) |
| 1 | 5 | 78.75% | 77.50% |
| 2 | 10 | 85.87% | 89.57% |
| 3 | 15 | 96.54% | 97.20% |
| 4 | 30 | 100.80% | 100.00% |
| 5 | 45 | 101.00% | 101.00% |
| 6 | 60 | 101.00% | 101.00% |

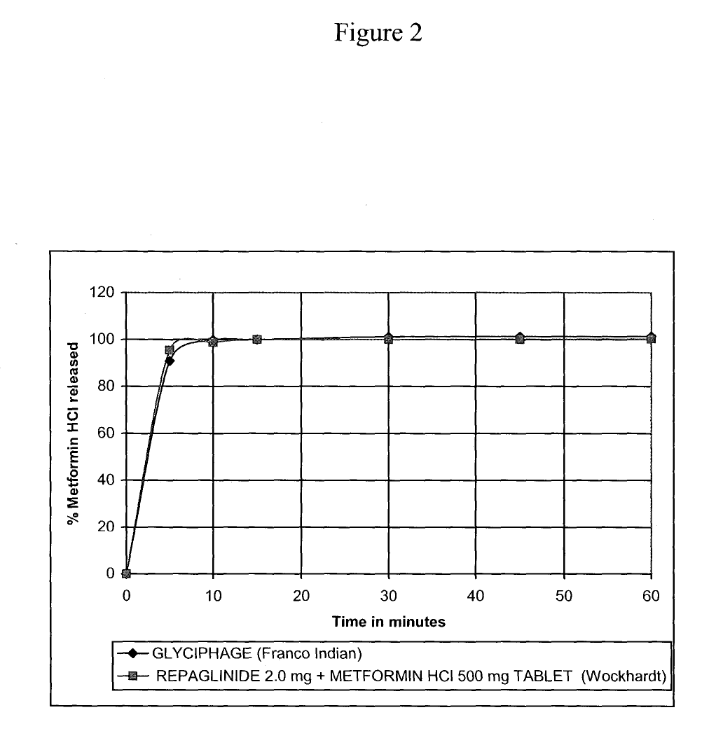

In addition, as can be seen in table 3 below, we next compared the dissolution profile for metformin from the unit-dose combination composition of the present invention (repaglinide+metformin (Wockhardt)) to that of metformin from its individually marketed form (GLYCIPHAGE™ (Franco Indian)). As shown in FIG. 2, which plots the data from table 3, the unit-dose combination composition of the present invention releases the metformin at substantially the same rate as per the individually marketed product.

TABLE 3
Comparative Dissolution Profile for Metformin HCl from
GLYCIPHAGE ™ (Franco Indian) and Repaglinide
2.0 mg + Metformin HCl 500 mg Tablets (Wockhardt)

| | Time | GLYCIPHAGE ™ | Repaglinide + Metformin |
|---|---|---|---|
| No. | (min) | (Franco Indian) | (Wockhardt) |
| 1 | 5 | 90.78% | 95.50% |
| 2 | 10 | 99.80% | 98.76% |
| 3 | 15 | 100.00% | 100.00% |
| 4 | 30 | 101.11% | 100.00% |
| 5 | 45 | 101.23% | 100.08% |
| 6 | 60 | 101.23% | 100.23% |

### Example 5

### Stability of the Novel Unit-Dose Combination Composition

The unit-dose combination composition of the present invention was subjected to accelerated stability testing by storing the product in controlled temperature chambers at 40° C. and 75% relative humidity for 3 months. There was no change in the physical properties of the tablets such as color and shape. Furthermore, there was no chemical degradation of the active agents as seen from the assay values at three months, i.e., 98.76% and 98.37% of the respective label claims of repaglinide and metformin.

Thus, this example, which employs repaglinide and metformin as the active agents, shows that the novel unit-dose combination composition of the present invention is physically and chemically stable under accelerated stability testing conditions of elevated temperature and humidity.

### Example 6

The following novel unit-dose combination compositions were also prepared in accordance with the present invention and demonstrated excellent content uniformity:

a) 0.5 mg of repaglinide and 500 mg of metformin: The tablets demonstrated an assay of repaglinide of 101.25% of label claim (0.5 mg per tablet) with a uniformity of content of 100.56%±1.76%, and an assay of metformin of 99.71% of label claim (500 mg per tablet).

b) 1 mg of repaglinide and 500 mg of metformin: The tablets demonstrated an assay of repaglinide of 101.18% of label claim (1 mg per tablet) with a uniformity of content of 99.91%±1.25%, and an assay of metformin of 99.84% of label claim (500 mg per tablet).

### Example 7

Similarly, unit-dose combination compositions of nateglinide and metformin could also be prepared by the techniques described herein, and in accordance with the present invention.

In the foregoing specification the invention has been described with reference to specific exemplary embodiments thereof. It will, however, be evident that various modifications and changes may be made thereto without departing from the broader spirit and scope of the invention as set forth in the appended claims. The specification should therefore be regarded in an illustrative rather than a restrictive sense.

We claim:

1. A unit-dose combination composition for the treatment of diabetes comprising:
a high-dose, water-soluble, long-acting, orally active, hypoglycemic, antidiabetic agent; and
a low-dose, water-insoluble, short-acting, orally active, hypoglycemic, antidiabetic agent;
wherein the low-dose agent and the high-dose agent are released simultaneously from the unit-dose combination composition, and wherein the low-dose agent is released at a rate which is substantially similar to a release rate of the low-dose agent in individual form, and the high-dose agent is released at a rate which is substantially similar to a release rate of the high-dose agent in individual form.

2. The composition of claim 1, wherein the high-dose agent comprises a member selected from the group consisting of a biguanide and a pharmaceutically acceptable salt thereof.

3. The composition of claim 2, wherein the high-dose agent comprises a member selected from the group consisting of metformin, a pharmaceutically acceptable salt of metformin, buformin, a pharmaceutically acceptable salt of buformin, phenformin, and a pharmaceutically acceptable salt of phenformin.

4. The composition of claim 3, wherein the high-dose agent comprises metformin present in an amount of from about 100 mg to about 2000 mg.

5. The composition of claim 4, wherein the high-dose agent comprises metformin present in an amount of from about 250 mg to about 1000 mg.

6. The composition of claim 1, wherein the low-dose agent comprises a member selected from the group consisting of a meglinitide and a pharmaceutically acceptable salt thereof.

7. The composition of claim 6, wherein the low-dose agent comprises a member selected from the group consisting of repaglinide and nateglinide.

8. The composition of claim 7, wherein the low-dose agent comprises repaglinide present in an amount of from about 0.20 mg to about 5.0 mg.

9. The composition of claim 8, wherein the low-dose agent comprises repaglinide present in an amount of from about 0.5 mg to about 2.0 mg.

10. The composition of claim 7, wherein the low-dose agent comprises nateglinide present in an amount of from about 20 mg to about 200 mg.

11. The composition of claim 10, wherein the low-dose agent comprises nateglinide present in an amount of from about 60 mg to about 180 mg.

12. The composition of claim 1, wherein the low-dose agent is distributed uniformly in the high-dose agent.

13. The composition of claim 1, wherein the low-dose agent and the high-dose agent are both in the form of an immediate release formulation.

14. The composition of claim 1, wherein the low-dose agent is an immediate-release component and the high-dose agent is a controlled-release component.

15. The composition of claim 14, wherein the high-dose agent comprises metformin, and the controlled release of the metformin is achieved by a mechanism selected from the group consisting of osmosis, diffusion, swelling, bioerosion, biodegradation and a combination of any of these principals.

16. A method of simultaneously administering repaglinide and metformin to a mammal, said method comprising:
administering to the mammal a unit-dose combination composition comprising repaglinide and metformin, wherein the repaglinide and the metformin are released simultaneously from the unit-dose combination composition, and wherein the repaglinide is released at a rate which is substantially similar to a release rate of the repaglinide in individual form, and the metformin is released at a rate which is substantially similar to a release rate of the metformin in individual form.

17. A method of simultaneously administering nateglinide and metformin to a mammal, said method comprising:
administering to the mammal a unit-dose combination composition comprising nateglinide and metformin, wherein the nateglinide and the metformin are released simultaneously from the unit-dose combination composition, and wherein the nateglinide is released at a rate which is substantially similar to a release rate of the nateglinide in individual form, and the metformin is released at a rate which is substantially similar to a release rate of the metformin in individual form.

18. A method of treating diabetes mellitus in a patient, said method comprising:
administering to the patient an effective amount of a unit-dose combination composition comprising:
a high-dose, water-soluble, long-acting, orally active, hypoglycemic, antidiabetic agent; and
a low-dose, water-insoluble, short-acting, orally active, hypoglycemic, antidiabetic agent;
wherein the low-dose agent and the high-dose agent are released simultaneously from the unit-dose combination composition, and wherein the low-dose agent is released at a rate which is substantially similar to a release rate of the low-dose agent in individual form, and the high-dose agent is released at a rate which is substantially similar to a release rate of the high-dose agent in individual form.
